(19) United States
(12) Patent Application Publication
(10) Pub. No.: US 2025/0174310 A1
(43) Pub. Date: May 29, 2025

(54) SYSTEM AND METHOD FOR CALCULATING MIXING CONDITION FOR DRY ELECTRODE

(71) Applicants: HYUNDAI MOTOR COMPANY, SEOUL (KR); KIA CORPORATION, SEOUL (KR)

(72) Inventors: Hyun Jin KIM, Daegu (KR); Yong Il CHO, Seoul (KR); Han Nah SONG, Suwon-si (KR)

(21) Appl. No.: 18/667,415

(22) Filed: May 17, 2024

(30) Foreign Application Priority Data
Nov. 24, 2023 (KR) 10-2023-0164997

(51) Int. Cl. G16C 20/20; G06V 10/75; G06V 20/69; H01M 4/04

(52) U.S. Cl. CPC G16C 20/20; G06V 10/75; G06V 20/698; H01M 4/043

(57) ABSTRACT

Disclosed is a dry electrode for secondary batteries. A system for calculating a mixing condition for a dry electrode includes a microscope configured to measure dispersion images of a first dry electrode mixture for each mixing condition, wherein an electrode active material, a conductive material, and a binder in the first dry electrode mixture are mixed by a mixer. A computing apparatus is configured to machine-learn the dispersion images of the first dry electrode mixture, to receive comparative dispersion images of a second dry electrode mixture, and to calculate a target mixing condition for the second dry electrode mixture based on machine-learned data of the dispersion images.

### CROSS-REFERENCE TO RELATED APPLICATION

This application claims under 35 U.S.C. § 119(a) the benefit of priority to Korean Patent Application No. 10-2023-0164997, filed on Nov. 24, 2023, the entire contents of which are incorporated herein by reference.

### TECHNICAL FIELD

The present disclosure relates to a dry electrode. More particularly, the present disclosure relates to a dry electrode for secondary batteries.

### BACKGROUND

Recently, application of rechargeable secondary batteries is expanding in various fields, from small electronic devices to large energy storage systems. In particular, research and development on secondary batteries is being actively conducted due to rapid growth of the electric vehicle market.

Electrodes for secondary batteries have generally been manufactured through a wet process. In the wet process, a slurry is manufactured by dissolving an electrode active material, a binder, and a conductive material included in the electrode with a solvent. However, more recently a dry process which may increase the energy density of a battery compared to the wet process without the solvent required in the wet process has been receiving a great deal of attention.

In the dry process for electrodes, a dry electrode film is prepared by preparing a mixture by mixing an electrode active material, a conductive material, and a binder, without any solvent, and then forming a film by pressing or calendaring. Then manufacture of an electrode may be completed by bonding the prepared dry electrode film to a current collector.

Compared to the wet electrode manufacturing process, the dry electrode manufacturing process may reduce manufacturing time and cost because no solvent is used, and may control the thickness of a film formed, thereby being capable of obtaining a dry electrode film having a high energy density.

However, in the dry process, as only electrode materials should be mixed without using a solvent, it is very difficult to control process conditions.

The above information disclosed in this Background section is only for enhancement of understanding of the background of the disclosure and therefore it may contain information that does not form the prior art that is already known to a person of ordinary skill in the art.

### SUMMARY OF THE DISCLOSURE

The present disclosure has been made in an effort to solve the above-described problems associated with the prior art, and it is an object of the present disclosure to provide a system for calculating a mixing condition for a dry electrode in which the mixing condition may be optimized regardless of the size of equipment used in the mixing process of the dry electrode and the amount of the mixed dry electrode.

The objects to be accomplished by the present disclosure are not limited to the above-mentioned objects, and other objects not mentioned herein will be clearly understood by those skilled in the art from the following description.

In one aspect, the present disclosure provides a system for calculating a mixing condition for a dry electrode includes a microscope configured to measure dispersion images of a first dry electrode mixture for each mixing condition, wherein an electrode active material, a conductive material, and a binder in the first dry electrode mixture are mixed by a mixer, and a computing apparatus configured to machine-learn the dispersion images of the first dry electrode mixture, and the computing apparatus is configured to receive comparative dispersion images of a second dry electrode mixture and to calculate a target mixing condition for the second dry electrode mixture based on machine-learned data of the dispersion images.

In another aspect, the present disclosure provides a method of calculating a mixing condition for a dry electrode including measuring, by a microscope, dispersion images of a first dry electrode mixture, in which an electrode active material, a conductive material and a binder are mixed by a mixer, under respective mixing conditions, machine-learning, by a computing apparatus, the dispersion images of the first dry electrode mixture, receiving, by the computing apparatus, comparative dispersion images of a second dry electrode mixture, and calculating, by the computing apparatus, a target mixing condition for the second dry electrode mixture based on machine-learned data of the dispersion images.

Other aspects and preferred embodiments of the disclosure are discussed infra.

The above and other features of the disclosure are discussed infra.

### BRIEF DESCRIPTION OF THE DRAWINGS

The above and other features of the present disclosure will now be described in detail with reference to certain exemplary embodiments thereof illustrated in the accompanying drawings which are given hereinbelow by way of illustration only, and thus are not limitative of the present disclosure, and wherein:

FIG. 1 is a view schematically illustrating a process of manufacturing a dry electrode;

FIG. 2 is a view schematically illustrating a mixer used in manufacture of the dry electrode;

FIG. 3 is a view illustrating a process of fibrillizing a binder while applying energy to materials forming a dry electrode;

FIG. 4A is a view illustrating changes in the state of the dry electrode depending on increase in energy applied to the materials forming the dry electrode;

FIG. 4B is a view illustrating changes in the state of the dry electrode depending on increase in energy applied to the materials forming the dry electrode;

FIG. 4C is a view illustrating changes in the state of the dry electrode depending on increase in energy applied to the materials forming the dry electrode;

FIG. 4D is a view illustrating changes in the state of the dry electrode depending on increase in energy applied to the materials forming the dry electrode;

FIG. 5 is a block diagram of a system for calculating a mixing condition for a dry electrode according to the present disclosure;

FIG. 6 is a view of an electrical conductivity measurer of the system according to one embodiment of the present disclosure;

FIG. 7 is a graph representing processing of data learned by a computing apparatus according to the present disclosure;

FIG. 8 is a diagram illustrating a machine learning model of the computing apparatus of the system according to the present disclosure; and

FIG. 9 is a graph representing a process of calculating an optimized mixing condition by the system according to the present disclosure.

It should be understood that the appended drawings are not necessarily to scale, presenting a somewhat simplified representation of various preferred features illustrative of the basic principles of the disclosure. The specific design features of the present disclosure as disclosed herein, including, for example, specific dimensions, orientations, locations, and shapes, will be determined in part by the particular intended application and use environment.

In the figures, reference numbers refer to the same or equivalent parts of the present disclosure throughout the several figures of the drawing.

### DETAILED DESCRIPTION

Specific structural or functional descriptions in embodiments of the present disclosure set forth in the description which follows will be exemplarily given to describe the embodiments of the present disclosure, and the present disclosure may be embodied in many alternative forms. Further, it will be understood that the present disclosure should not be construed as being limited to the embodiments set forth herein, and the embodiments of the present disclosure are provided only to completely disclose the disclosure and cover modifications, equivalents or alternatives which come within the scope and technical range of the disclosure.

In the following description of the embodiments, terms, such as “first” and “second”, are used only to describe various elements, and these elements should not be construed as being limited by these terms. These terms are used only to distinguish one element from other elements. For example, a first element described hereinafter may be termed a second element, and similarly, a second element described hereinafter may be termed a first element, without departing from the scope of the disclosure.

When an element or layer is referred to as being “connected to” or “coupled to” another element or layer, it may be directly connected or coupled to the other element or layer, or intervening elements or layers may be present. In contrast, when an element or layer is referred to as being “directly connected to” or “directly coupled to” another element or layer, there may be no intervening elements or layers present. Other words used to describe relationships between elements should be interpreted in a like fashion, e.g., “between” versus “directly between,” “adjacent” versus “directly adjacent,” etc.

Wherever possible, the same reference numbers will be used throughout the drawings to refer to the same or like parts. The terminology used herein is for the purpose of describing particular embodiments only and is not intended to be limiting. As used herein, singular forms may be intended to include plural forms as well, unless the context clearly indicates otherwise. The terms “comprises,” “comprising,” “including,” and “having” are inclusive and therefore specify the presence of stated features, integers, operations, operations, elements, components, and/or combinations thereof, but do not preclude the presence or addition of one or more other features, integers, operations, operations, elements, components, and/or combinations thereof.

Hereinafter, reference will be made in detail to various embodiments of the present disclosure, examples of which are illustrated in the accompanying drawings and described below.

A dry electrode may be manufactured from a dry electrode mixture M and a current collector without a solvent. The dry electrode mixture M is a mixture including an electrode active material, a conductive material, and a binder. Further, the dry electrode mixture M may further include additives.

The dry electrode may be a cathode or may be an anode. In some embodiments, when a cathode is manufactured, the electrode active material may include a cathode active material. As a non-limiting example, the cathode active material may include LiCoO₂ (LCO), Li(Ni,Co,Mn)O₂ (NCM), Li(Ni,Co,Al)O₂ (NCA), LiMnO₄ (LMO), LiFePO₄ (LFP), or sulfur (S).

In some embodiments, when an anode is manufactured, the electrode active material may include an anode active material. As a non-limiting example, the anode active material may include natural graphite, artificial graphite, mesocarbon microbeads (MCMB), or a silicon-based material.

The conductive material may include a carbon material. For example, the conductive material may include carbon black, acetylene black, carbon fibers, or carbon nanotubes.

The binder may include a polymer-based chemical, such as polyvinylidene fluoride (PVDF), polyvinyl alcohol (PVA), polytetrafluoroethylene (PTFE), styrene-butadiene rubber (SBR), carboxymethyl cellulose (CMC), polyacrylonitrile (PAN), or the like.

As additives, some of a solid polymer electrolyte, such as poly (ethylene oxide) (PEO), or oxide and sulfide-based solid electrolytes may be used.

The dry electrode mixture M may include 70 to 99.9 wt % of the electrode active material, 0.1 to 20 wt % of the conductive material, and 0.1 to 20 wt % of the binder, as dry electrode materials. Here, the additives may be added at a rate of 0 to 20 wt %.

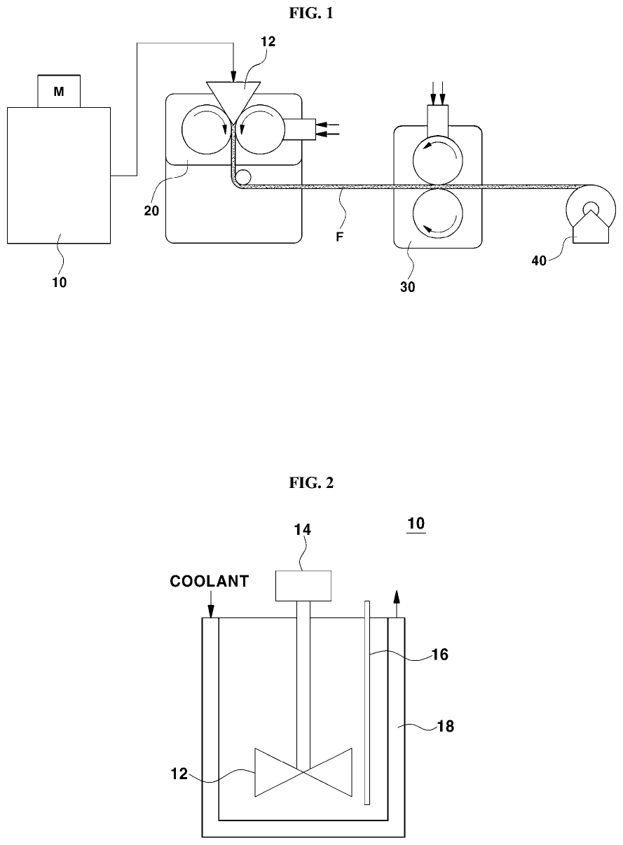

As shown in FIG. 1, the dry electrode mixture M may be manufactured into a dry electrode film F through a series of film formation processes in which heat and pressure are applied. First, the dry electrode mixture M including the electrode active material, the conductive material, and the binder is mixed at a predetermined speed for a predetermined time by a mixture 10. As a non-limiting example, the dry electrode mixture M may be manufactured through a high shear mixer using rotation or a fluid mixer using air, and the predetermined time and speed may be adjusted through changes in the rotational speed and operating time of the mixer 10. As another non-limiting example, the dry electrode mixture M may be manufactured using a compactor, a granulator, or a combination thereof.

The mixed dry electrode mixture M may be primarily pressed into a film by an upstream roll press 20. The upstream roll press 20 rotates while pressing the dry electrode mixture M to form the dry electrode mixture M into the film. The dry electrode film F may be additionally pressed by a downstream roll press 30 so that the thickness of the dry electrode film may be adjusted through pressing. The obtained dry electrode film F is wound on a winder 40. Thereafter, a dry electrode may be manufactured by bonding or laminating the dry electrode film F to or on the current collector. Formation of the dry electrode film F and lamination of the dry electrode film F on the current collector may be performed in one apparatus or may be respectively performed in separate apparatuses.

In the manufacturing process of the dry electrode, mixing of the dry electrode mixture M may be performed through the mixer 10. In some embodiments, in such a mixing process, the dry electrode mixture M may be mixed by dispersing the electrode active material and the conductive material and then adding the binder thereto. In some embodiments, the dry electrode mixture M may be mixed by dispersing the electrode active material, the conductive material, and the binder together.

In some embodiments, the mixer 10 may include a spiral mixer, a vertical mixer, a horizontal mixer, an oblique mixer, a planetary mixer, a paddle mixer, a screw mixer, a stand mixer, a granulator, a jet mill, a compactor, or the like. In some embodiments, mixing may be performed through two or more mixers selected therefrom. However, the mixer 10 is not limited thereto.

As mixing conditions, a temperature in the range of −20° C. to 200° C. may be used. In some embodiments, the mixer 10 may further include a chiller so that a low temperature may be maintained during mixing.

FIG. 2 shows an exemplary mixer 10. The mixer 10 includes a rotatable blade 12. The blade 12 may be driven by a driver 14, such as an electric motor. The mixer 10 may further include a feed 16. The feed 16 is provided outside the blade 12, and may scrape materials only rotating without being mixed so that shearing force is evenly applied to electrode materials forming a dry electrode put into the mixer 16.

The mixer 10 may further include a cooling jacket 18. A coolant circulated through the cooling jacket 18 may provide control of potential temperature changes due to heat generated during mixing.

When the materials forming the dry electrode mixture M, i.e., the electrode active material, the conductive material, and the binder (additionally, the additives), are put into the mixer 10 and the blade 12 is rotated, energy is transferred to respective particles of the materials. When the binder in the materials acquires appropriate energy through mixing and becomes fibrillized, the mixing process may be completed as the fibrillized binder connects the active material, the conductive material and the additives into a network.

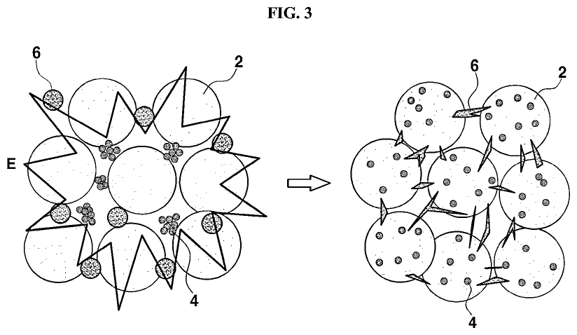

As shown in FIG. 3, when the electrode materials are put into the mixer 10, energy E is applied to the electrode materials in various ways depending on the type of the mixer 10. Due to this energy E, particles of the conductive material 4 gathered in the form of clusters are physically dispersed and combined with the surfaces of particles of the electrode active material 2. The binder 6 may be fibrillized to form the network among the particles of the materials. Here, the energy E applied to the materials may be resulted from various factors, such as impact applied by the blade 12, collisions between air and the particles, etc. When the mixer 10 is set, the amount of energy applied to the electrode materials may be adjusted through the mixing time and/or mixing speed of the mixer 10.

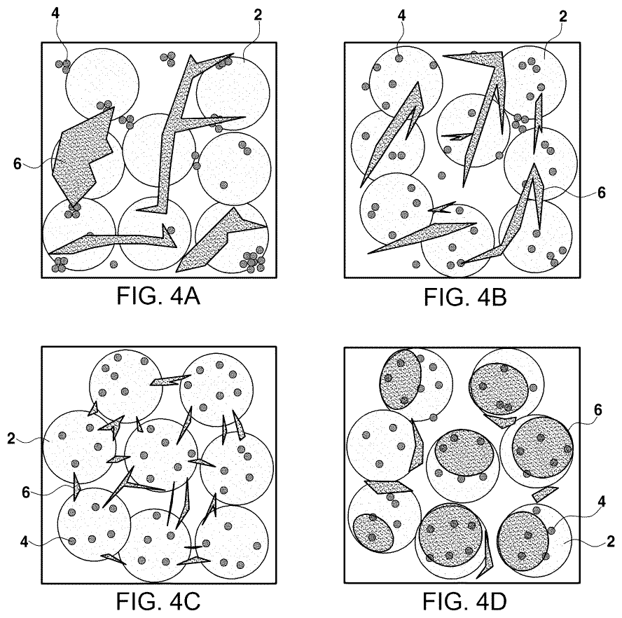

Specifically, the mixing process of the dry electrode mixture M may be divided into four steps, as shown in FIGS. 4A, 4B, 4C, and 4D. As the energy (i.e., the mixing time and/or mixing speed) applied to the electrode materials increases, steps shown in FIGS. 4A, 4B, 4C, and 4D may take place in sequence.

As shown in FIG. 4A, the dry electrode materials are put into the mixer 10, and the binder 6 is in a clumped state at an initial stage of mixing. The binder 6 includes primary particles and secondary particles. The primary particles refer to particles of 500 nanometers or less which form the binder 6, and particles formed by aggregation of a plurality of these primary particles to a size of hundreds of micrometers may be referred to as the secondary particles. In the state shown in FIG. 4A, due to insufficient energy applied to the electrode materials, the binder 6 is not sufficiently transformed into primary particles and is aggregated in the state of secondary particles. The conductive material 4 is also not sufficiently dispersed and is in a clumped state.

FIG. 4B shows a state in which the binder 6 begins to be fibrillized. In this state, as the energy (i.e., the mixing time and/or mixing speed) applied to the respective particles of the materials increases, fibrillization of the binder 6 occurs. Separation of the secondary particles of the binder 6 into primary particles occurs, and thus, some of particles of the binder 6 undergo fibrillization (i.e., have the shape of a skein) but other particles of the binder 6 remain in the state of secondary particles.

FIG. 4C shows a state in which fibrillization of the binder 6 has been appropriately completed and shows a target state to be achieved through mixing of the dry electrode mixture M. As the binder 6 is separated into the primary particles, fibrillization of the binder 6 occurs. The conductive material 4 is also uniformly disposed on the surfaces of the particles of the electrode active material 2.

FIG. 4D shows a particle coating state in which hyper fibrillization of the binder 6 occurs as excess energy is applied to the electrode materials. Here, the surfaces of the particles of the electrode active material 2 coated with the binder 6 are observed. The network formed among the particles of the materials through fibrillization of the binder 6 disappears, which decreases possibility of forming a film.

Although the ratio of the electrode materials and operating conditions of the mixer 10 to obtain the dry electrode mixture M in a satisfactory fibrillized state of the binder 6 are obtained, as shown in FIG. 4(c), when the scale of mixing is changed or the amounts of the electrode materials put into the mixer 10 are changed, the ratio of the electrode materials and the operating conditions of the mixer 10 should be changed. This is partly because the dry process involves fibrillizing the binder 6 by applying appropriate energy to the binder 6 without using a solvent.

After a target mixing time and target mixing speed for the dry electrode mixture M are obtained in the mixer 10 having a small capacity, e.g., a capacity of 10 L, a verification is made on a small scale. When the verified small-scale data is modified to suit a large scale for mass production, it was found that the same results could not be obtained. This is because energy at which particles collide is changed even though the mixer 10 is rotated at the same linear speed.

In addition, when the input amounts of the electrode materials changes, the amount of collision of the particles of the electrode materials per hour is changed. This causes the energy applied to the particles to change, desired results may not be obtained with the same process as in the small scale.

Certainly, the collision energy applied to the particles may be calculated through simulation. However, a large error between simulation results and the results of the actual system is generated due to presence of diverse factors in the actual system. As such, the dry process requires a lot of time and cost to determine appropriate mixing conditions, and thus, the present disclosure suggests a method of optimizing a mixing process for a dry electrode through machine learning.

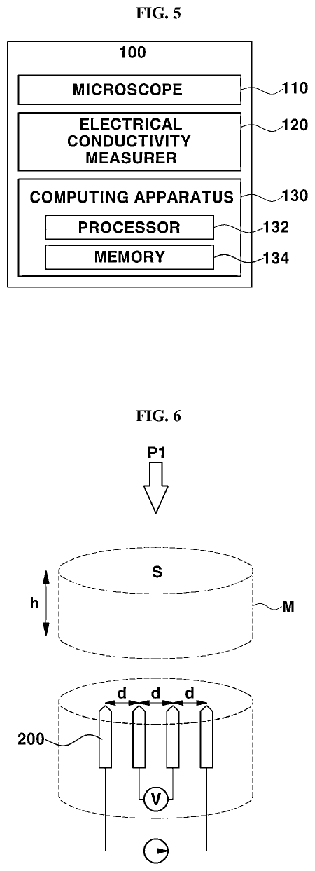

Referring to FIG. 5, a system 100 for calculating a mixing condition for a dry electrode according to the present disclosure includes a microscope 110, an electrical conductivity measurer 120, and a computing apparatus 130.

The microscope 110 may measure dispersion images of the dry electrode mixture M. The microscope 110 may be an optical microscope or an electron microscope. For example, the electron microscope may be a scanning electron microscope (SEM) or a transmission electron microscope (TEM), but is not limited thereto. The magnification of the microscope 110 may be adjusted. However, as will be described below, the microscope 110 is configured such that dispersion images for machine learning of the computing apparatus 130 are measured at the same magnification.

As shown in FIG. 6, the electrical conductivity measurer 120 may measure the electrical conductivity of the dry electrode mixture M by applying a pressure or force P1 to a designated amount of the dry electrode mixture M having an area S and a height h. The force P1 may have a value, for example, selected between 1 kilonewton (kN) and 100 kN. The electrical conductivity may be measured by a probe 200 positioned at the lower end of the dry electrode mixture M. Here, the probe 200 may be a 4-point probe. The electrical conductivity (Siemens per centimeter, S/cm) of the dry electrode mixture M with respect to the force P1 applied by the electrical conductivity measurer 120 may be measured. The force P1 may be changed, and electrical conductivity values of the dry electrode mixture M may be measured at respective magnitudes of the force P1. As will be described below, the computing apparatus 130 may learn the average value of the electrical conductivity values at the respective magnitudes of the force P1 and may calculate a mixing time or a mixing speed based on the learned data.

The computing apparatus 130 may collect data and learn the collected data. In one embodiment, the computing apparatus 130 may collect the dispersion image and electrical conductivity data of the dry electrode mixture M. In some embodiments, the dispersion image and electrical conductivity of the dry electrode mixture M are obtained for each mixing time by varying the mixing time of the dry electrode mixture M. At this time, the mixing speed is fixed. In another embodiment, the dispersion image and electrical conductivity of the dry electrode mixture M are obtained for each mixing speed by varying the mixing speed of the dry electrode mixture M. At this time, the mixing time is fixed. In addition, the dispersion image and electrical conductivity of the dry electrode mixture M are obtained for each type and ratio of the respective materials forming the dry electrode mixture M.

The computing apparatus 130 may machine-learn the collected data. The computing apparatus 130 may execute machine learning algorithms and may be trained using a machine learning model. As a non-limiting example, logistic regression, random forests, neural networks, or the like may be used as the machine learning model.

The computing apparatus 130 may acquire low-dimensional feature vectors for the dispersion images of the dry electrode mixture M through image embedding. As a non-limiting example, Inception V3 may be used as an image embedder. The machine learning model of the computing apparatus 130 may use the feature vectors of the respective images as inputs. Here, as the dispersion images, data taken at the same magnification by the microscope 110 having the same conditions is learned by the computing apparatus 130. For example, the magnification may be at least 500×.

The electrical conductivities of the dry electrode mixture M at the respective magnitudes of the force P1 are measured by the electrical conductivity measurer 120, and the average value thereof is learned by the computing apparatus 130. For example, one value which is the average value of 10 electrical conductivities (S/cm) measured at 1, 2, 3, 4, 5, 6, 7, 8, 9, and 10 kN corresponds to one mixing time or mixing speed. The force when measuring electrical conductivity may be set to various magnitudes, but the averages of electrical conductivities measured under the same pressure or force conditions are learned by the computing apparatus 130.

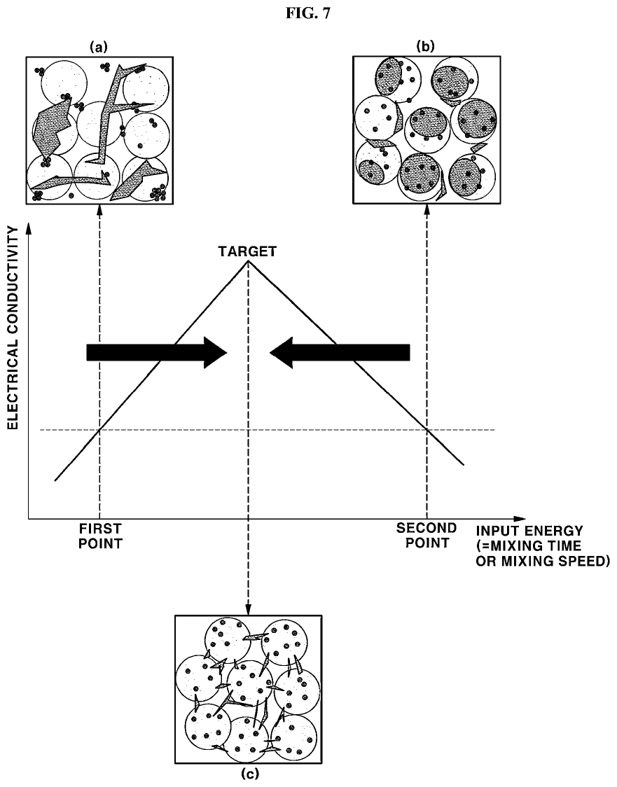

Electrical conductivity depending on a mixing time or mixing speed may be obtained in the form of a graph, as shown in FIG. 7. The electrical conductivity of the dry electrode mixture M may be obtained at each input energy (i.e., each mixing time or mixing speed) where the electrical conductivity may be an average value in a designated range of force applied, as described above. Further, dispersion image data at each position in the graph may be matched to corresponding electrical conductivity and may be used together.

In electrical conductivity data depending on the mixing time or mixing speed, there are parts having the same value or parts having values overlapping each other. In this respect, the entire section should be examined to determine whether the dry electrode mixture M reaches the satisfactory level of fibrillization of the binder based on electrical conductivity alone. Therefore, the present disclosure trains the computing apparatus 130 with both the electrical conductivity data and dispersion data as feature values to clearly distinguish a first point and a second point from each other.

As shown in FIG. 8, according to the present disclosure, the computing apparatus 130 may receive the dispersion image and electrical conductivity of a target dry electrode mixture, which requires calculation of an optimized mixing condition, as inputs. Then the computing apparatus 130 may calculate the optimized mixing condition, i.e., a mixing time or a mixing speed, as outputs. This will be described below through an example.

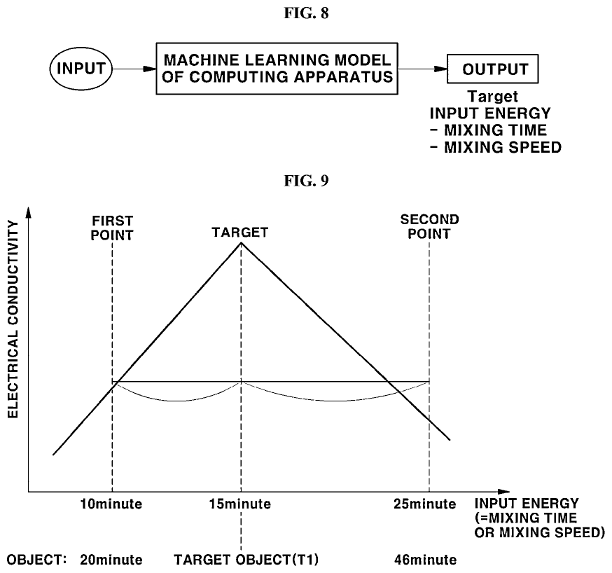

Referring to FIG. 9, it is assumed that, in data regarding a 10 kilogram (kg) mixture M learned by the computing apparatus 130, a mixing time at a first point is 10 minutes, a mixing time at a target point where a binder is properly fibrillized is 15 minutes, and a mixing time at a second point is 25 minutes. In the following example, the linear speed of the blade 12 is fixed to 30 m/s.

A first target dry electrode mixture which requires calculation of an optimized mixing condition is prepared. The input amount of the first target dry electrode mixture is changed to 20 kg. After mixing the first target dry electrode mixture for a preset first mixing time (e.g., 20 minutes), a first dispersion image and a first electrical conductivity of the first target dry electrode mixture are measured. When the first dispersion image and the first electrical conductivity are input to the computing apparatus 130, a corresponding position in the data shown in FIG. 9 may be determined. As in the illustrated embodiment, it is assumed that the corresponding position is the position of the first point.

Thereafter, a second target dry electrode mixture is prepared. The components and the composition ratio thereof of the second target dry electrode mixture are the same as those of the first target dry electrode mixture, but the second target dry electrode mixture is mixed for a second mixing time (e.g., 46 minutes) different from the first mixing time. Thereafter, a second dispersion image and a second electrical conductivity of the second target dry electrode mixture are measured. When the second dispersion image and the second electrical conductivity are input to the computing apparatus 130, a corresponding position in the data shown in FIG. 9 may be determined. As in the illustrated embodiment, it is assumed that the corresponding position is the position of the second point.

Through the results of the first target dry electrode mixture and the second target dry electrode mixture corresponding to the first point and the second point, the computing apparatus 130 may calculate a ratio with the target point and may calculate an optimized mixing time in the changed process. That is, the computing apparatus 130 may calculate the optimized mixing time of 28.67 minutes as a target object through Equation 20 mins+(46 mins−20 mins)×5/(25 mins−10 mins).

This calculation process may be equally applied to a mixing speed. When calculating the mixing speed, the mixing time is set to be the same, whereas when calculating the mixing time, the mixing speed is set to be the same. When the mixing time is a target, the mixing speed of the mixing conditions is fixed based on the linear speed of the mixer 10. Further, when the mixing speed is a target, the mixing time of the mixing conditions is fixed to a certain time as long as fibrillization of the binder is possible.

The computing apparatus 130 includes a processor 132 and a memory 134. Instructions executable by the computing apparatus 130 are stored in the memory 134. In some embodiments of the present disclosure, the instructions may include instructions for executing operation of the computing apparatus 130 and/or operations of the respective components of the computing apparatus 130.

The memory 134 may be a volatile or non-volatile memory. As a non-limiting example, the volatile memory may be a dynamic random access memory (DRAM), a static random access memory (SRAM), or the like. As another non-limiting example, the non-volatile memory may be an electrically erasable programmable read-only memory (EEPROM), a flash memory, a magnetic RAM (MRAM), a CD-ROM, a DVD-ROM, or the like.

Further, the memory 134 may store a matrix on which operations included in the machine learning model are to be performed, and the memory 134 may store operation results generated by processing of the computing apparatus 130.

The processor 132 may execute the instructions stored in the memory 134. The processor 132 may execute computer-readable code and instructions stored in the memory 134. As a non-limiting example, the processor 132 may include a central processing unit, a graphics processing unit, a neural processing unit, a multi-core processor, a multiprocessor, an application-specific integrated circuit (ASIC), or a field programmable gate array (FPGA).

According to some embodiments of the present disclosure, the system 100 may be implemented in the form of a recording medium including instructions executable by computers, such as program modules executed by computers. Computer-readable media may be any available media accessible by computers and include volatile and non-volatile media, and removable and non-removable media. Additionally, computer-readable media may include all computer storage media. Computer storage media include volatile and nonvolatile media, and removable and non-removable media implemented in any method or technology for storage of information such as computer-readable instructions, data structures, program modules or other data.

The present disclosure may reduce test materials and time consumed to optimize the mixing process of a dry electrode and may effectively respond to even a situation where conditions should be changed in various ways.

As is apparent from the above description, the present disclosure provides a system for calculating a mixing condition for a dry electrode in which the mixing condition may be optimized regardless of the size of equipment used in the mixing process of the dry electrode and the amount of the mixed dry electrode.

The effects of the present disclosure are not limited to the above-mentioned effects, and other effects not mentioned herein will be clearly understood by those skilled in the art from the above description.

The disclosure has been described in detail with reference to preferred embodiments thereof. However, it will be appreciated by those skilled in the art that changes may be made in these embodiments without departing from the principles and spirit of the disclosure, the scope of which is defined in the appended claims and their equivalents.

What is claimed is:

1. A system for calculating a mixing condition for a dry electrode comprising:
a microscope configured to measure dispersion images of a first dry electrode mixture for each mixing condition, wherein an electrode active material, a conductive material and a binder in the first dry electrode mixture are mixed by a mixer; and
a computing apparatus configured to machine-learn the dispersion images of the first dry electrode mixture,
wherein the computing apparatus is configured to receive comparative dispersion images of a second dry electrode mixture and to calculate a target mixing condition for the second dry electrode mixture based on machine-learned data of the dispersion images.

2. The system of claim 1, wherein the mixing condition is a mixing time or a mixing speed by the mixer.

3. The system of claim 2, wherein:
when the mixing condition is the mixing time by the mixer, linear speeds of mixers used to mix the first dry electrode mixture and the second dry electrode mixture are set to be the same; or
when the mixing condition is the mixing speed by the mixer, operating times of the mixers used to mix the first dry electrode mixture and the second dry electrode mixture are set to be the same.

4. The system of claim 1, wherein the target mixing condition is a mixing time or a mixing speed by the mixer when a binder included in the second dry electrode mixture satisfies predetermined fibrillization conditions.

5. The system of claim 1, wherein:
components and composition ratios of the first dry electrode mixture and the second dry electrode mixture are the same, but amounts of the first dry electrode mixture and the second dry electrode mixture are different; or
the components and the composition ratios of the first dry electrode mixture and the second dry electrode mixture are the same, but apparatuses for respectively mixing the first dry electrode mixture and the second dry electrode mixture are different.

6. The system of claim 1, wherein:
the machine-learned data comprises a comparative mixing condition which is a mixing condition when the binder in the first dry electrode mixture satisfies predetermined fibrillization conditions; and
the computing apparatus is configured to:
acquire a first comparative dispersion image of the second dry electrode mixture mixed through a first comparative mixing time;
acquire a first mixing time which is a mixing time of the first dry electrode mixture when the first comparative dispersion image corresponds to one of the dispersion images of the first dry electrode mixture;
acquire a second comparative dispersion image of the second dry electrode mixture mixed through a second comparative mixing time;
acquire a second mixing time which is a mixing time of the first dry electrode mixture when the second comparative dispersion image corresponds to another of the dispersion images of the first dry electrode mixture; and
determine the target mixing condition for the second dry electrode mixture based on a ratio of the first mixing time, the second mixing time and the comparative mixing condition.

7. The system of claim 1, wherein:
the machine-learned data comprises a comparative mixing condition which is a mixing condition when the binder in the first dry electrode mixture satisfies predetermined fibrillization conditions; and
the computing apparatus is configured to:
acquire a first comparative dispersion image of the second dry electrode mixture mixed through a first comparative mixing speed;
acquire a first mixing speed which is a mixing speed of the first dry electrode mixture when the first comparative dispersion image corresponds to one of the dispersion images of the first dry electrode mixture;
acquire a second comparative dispersion image of the second dry electrode mixture mixed through a second comparative mixing speed;
acquire a second mixing speed which is a mixing speed of the first dry electrode mixture when the second comparative dispersion image corresponds to another of the dispersion images of the first dry electrode mixture; and
determine the target mixing condition for the second dry electrode mixture based on a ratio of the first mixing speed, the second mixing speed and the comparative mixing condition.

8. The system of claim 1, further comprising an electrical conductivity measurer configured to measure electrical conductivities of the first dry electrode mixture under the respective mixing conditions,
wherein the computing apparatus is configured to:
further machine-learn the electrical conductivities;
further receive comparative electrical conductivities of the second dry electrode mixture; and
calculate the target mixing condition for the second dry electrode mixture based on the machine-learned data of the dispersion images and the electrical conductivities.

9. The system of claim 8, wherein electrical conductivity values are measured while pressing the first dry electrode mixture or the second dry electrode mixture at respective pressures under each of the mixing conditions, and each of the electrical conductivities is an average value of the electrical conductivity values obtained at the respective pressures.

10. The system of claim 8, wherein the machine-learned data comprises a comparative mixing condition which is a mixing condition when the binder in the first dry electrode mixture satisfies predetermined fibrillization conditions.

11. The system of claim 10, wherein the computing apparatus is configured to:
acquire a first comparative dispersion image and first comparative electrical conductivity of the second dry electrode mixture mixed through a first comparative mixing time;
acquire a first mixing time which is a mixing time of the first dry electrode mixture when the first comparative dispersion image and the first comparative electrical conductivity correspond to one of the dispersion images and one of the electrical conductivities of the first dry electrode mixture;
acquire a second comparative dispersion image and second comparative electrical conductivity of the second dry electrode mixture mixed through a second comparative mixing time;
acquire a second mixing time which is a mixing time of the first dry electrode mixture when the second comparative dispersion image and the second comparative electrical conductivity correspond to another of the dispersion images and another of the electrical conductivities of the first dry electrode mixture; and
determine the target mixing condition for the second dry electrode mixture based on a ratio of the first mixing time, the second mixing time and the comparative mixing condition.

12. The system of claim 10, wherein the computing apparatus is configured to:
acquire a first comparative dispersion image and first comparative electrical conductivity of the second dry electrode mixture mixed through a first comparative mixing speed;
acquire a first mixing speed which is a mixing speed of the first dry electrode mixture when the first comparative dispersion image and the first comparative electrical conductivity correspond to one of the dispersion images and one of the electrical conductivities of the first dry electrode mixture;
acquire a second comparative dispersion image and second comparative electrical conductivity of the second dry electrode mixture mixed through a second comparative mixing speed;
acquire a second mixing speed which is a mixing speed of the first dry electrode mixture when the second comparative dispersion image and the second comparative electrical conductivity correspond to another of the dispersion images and another of the electrical conductivities of the first dry electrode mixture; and
determine the target mixing condition for the second dry electrode mixture based on a ratio of the first mixing speed, the second mixing speed and the comparative mixing condition.

13. A method of calculating a mixing condition for a dry electrode comprising:
measuring, by a microscope, dispersion images of a first dry electrode mixture, in which an electrode active material, a conductive material and a binder are mixed by a mixer, under respective mixing conditions;
machine-learning, by a computing apparatus, the dispersion images of the first dry electrode mixture;
receiving, by the computing apparatus, comparative dispersion images of a second dry electrode mixture; and
calculating, by the computing apparatus, a target mixing condition for the second dry electrode mixture based on machine-learned data of the dispersion images.

14. The method of claim 13, further comprising:
measuring, by an electrical conductivity measurer, electrical conductivities of the first dry electrode mixture under the respective mixing conditions;
additionally machine-learning, by the computing apparatus, the electrical conductivities; and
receiving, by the computing apparatus, comparative electrical conductivities of the second dry electrode mixture,
wherein calculating the target mixing condition comprises calculating the target mixing condition based on machine-learned data of the electrical conductivities.

15. The method of claim 14, wherein the machine-learned data comprises a comparative mixing condition which is a mixing condition when the binder in the first dry electrode mixture satisfies predetermined fibrillization conditions,
wherein the method further comprises:
acquiring, by the computing apparatus, a first comparative dispersion image and first comparative electrical conductivity of the second dry electrode mixture mixed through a first comparative mixing time;
acquiring, by the computing apparatus, a first mixing time which is a mixing time of the first dry electrode mixture when the first comparative dispersion image and the first comparative electrical conductivity correspond to one of the dispersion images and one of the electrical conductivities of the first dry electrode mixture;
acquiring, by the computing apparatus, a second comparative dispersion image and second comparative electrical conductivity of the second dry electrode mixture mixed through a second comparative mixing time;
acquiring, by the computing apparatus, a second mixing time which is a mixing time of the first dry electrode mixture when the second comparative dispersion image and the second comparative electrical conductivity correspond to another of the dispersion images and another of the electrical conductivities of the first dry electrode mixture; and
determining, by the computing apparatus, the target mixing condition for the second dry electrode mixture based on a ratio of the first mixing time, the second mixing time and the comparative mixing condition.

16. The method of claim 14, wherein the machine-learned data comprises a comparative mixing condition which is a mixing condition when the binder in the first dry electrode mixture satisfies predetermined fibrillization conditions,
wherein the method further comprises:
acquiring, by the computing apparatus, a first comparative dispersion image and first comparative electrical conductivity of the second dry electrode mixture mixed through a first comparative mixing speed;
acquiring, by the computing apparatus, a first mixing speed which is a mixing speed of the first dry electrode mixture when the first comparative dispersion image and the first comparative electrical conductivity correspond to one of the dispersion images and one of the electrical conductivities of the first dry electrode mixture;
acquiring, by the computing apparatus, a second comparative dispersion image and second comparative electrical conductivity of the second dry electrode mixture mixed through a second comparative mixing speed;
acquiring, by the computing apparatus, a second mixing speed which is a mixing speed of the first dry electrode mixture when the second comparative dispersion image and the second comparative electrical conductivity correspond to another of the dispersion images and another of the electrical conductivities of the first dry electrode mixture; and
determining, by the computing apparatus, the target mixing condition for the second dry electrode mixture based on a ratio of the first mixing speed, the second mixing speed and the comparative mixing condition.

17. The method of claim 14, wherein electrical conductivity values are measured while pressing the first dry electrode mixture or the second dry electrode mixture at respective pressures under each of the mixing conditions, and each of the electrical conductivities is an average value of the electrical conductivity values obtained at the respective pressures.

18. The method of claim 13, wherein:
the mixing condition is a mixing time or a mixing speed by the mixer; and
the target mixing condition is a mixing time or a mixing speed by the mixer when a binder comprised in the second dry electrode mixture satisfies predetermined fibrillization conditions.

19. The method of claim 13, wherein:
components and composition ratios thereof of the first dry electrode mixture and the second dry electrode mixture are the same, but amounts of the first dry electrode mixture and the second dry electrode mixture are different; or
the components and the composition ratios thereof of the first dry electrode mixture and the second dry electrode mixture are the same, but apparatuses for respectively mixing the first dry electrode mixture and the second dry electrode mixture are different.

20. The method of claim 13, wherein:
when the mixing condition is a mixing time by the mixer, linear speeds of mixers used to mix the first dry electrode mixture and the second dry electrode mixture are set to be the same; and
when the mixing condition is a mixing speed by the mixer, operating times of the mixers used to mix the first dry electrode mixture and the second dry electrode mixture are set to be the same.
